(19) United States
(12) Patent Application Publication
(10) Pub. No.: US 2008/0103522 A1
(43) Pub. Date: May 1, 2008

(54) Distal Protection Device for Filtering and Occlusion

(75) Inventors: Allan Steingisser, Melrose, MA (US); Paul Squadrito, Rowley, MA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/553,844

(22) Filed: Oct. 27, 2006

(51) Int. Cl. A61M 29/00

(52) U.S. Cl. 606/200

(57) ABSTRACT

A distal protection device for capturing embolic debris during a vascular interventional procedure. The distal protection device is selectively transformable between a filtration configuration, which allow perfusion while capturing embolic debris, and an occlusive configuration, which blocks blood flow through the device and thereby traps embolic debris for subsequent removal. The distal protection device includes a filter component and a coil occluder component. The coil occluder component includes windings that may be advanced or withdrawn from within the filter component, the windings being selectively stackable within the filter component to occlude blood flow through the filter component. Methods of using the distal protection device are also disclosed.

### FIELD OF THE INVENTION

The present invention related to distal protection devices. More particularly, the invention relates to a convertible filtration and occlusion device for capturing and removing emboli in a blood vessel during an interventional vascular procedure.

### BACKGROUND OF THE INVENTION

Diseased blood vessels are a widespread medical condition. For example, atherosclerotic plaque may develop in blood vessel walls, a thrombus (blood clot) may form in a vessel, or a stenosis may form. If a blood vessel becomes weakened, or if the accumulation of plaque or thrombi on blood vessel walls becomes too server, surgical intervention may be required to prevent rupture or complete occlusion of the vessels. While many different surgical procedures are associated with alleviating this condition, the use of catheters is preferred, due to the minimally invasive nature of procedures involving catheters.

Many types of procedures involve the use of catheters to treat stenotic vessels or thromboses. One type of procedure is percutaneous transluminal coronary angioplasty, or PTCA, which involves the inflation of an angioplasty balloon catheter in a stenosis to dilate a coronary blood vessel. Additionally, a stent may be implanted in conjunction with this procedure to prevent restenosis, or re-narrowing of the vessel. Various other catheter-based procedures are also common, such as thrombectomy to remove a thrombus or a portion thereof or atherectomy to cut out or abrade a stenosis within a diseased portion of the vessel.

Each of these modalities is associated with a risk that particles will be dislodged during the procedure and migrate through the circulatory system to embolize, possibly causing ischaemia, infarction or stoke. To prevent patient injury from such loosened debris, clinicians may attempt to capture the potentially embolic particles using occlusion devices or embolic filers, then lysing or aspirating the entrapped particles, or removing the particles along with the filter.

Each of these embolic protection devices and methods has certain advantages and certain drawbacks. Occlusion devices will prevent all of the loosened embolic material from migrating. However, since an occluder also prevents blood flow, the duration of use of an occluder is limited. As such, occlusion is not appropriate in all cases. Further, removal of the embolic particles caught by the occluder typically requires an additional step, such as aspiration, sometimes by insertion of an additional aspiration device into the treated vessel.

Embolic filters may be used for longer duration than occluders because filtering devices do not prevent the flow of fluid. Thus, filter devices may be used in a wider variety of procedures, although embolic filters also suffer from some drawbacks. Filters are limited in their ability to remove very small embolic particles from the bloodstream. Additionally, an embolic filter may fill up with debris sufficiently for the filer to occlude the vessel unless the filter is removed or emptied in-situ by aspiration.

A combination of filters and occluders on the same catheter has been proposed for use in heart surgery where the heart must be arrested and isolated from the rest of the cardiovascular system. One such combination filter and occluder includes a blood filtration assembly for filtering blood and a balloon occlude. However, in such devices, the filter and occluder are generally spatially separated along the shaft of a cannula such that the occluder is positioned upstream of the filter. The separation of the filter and occluder structures is often not practical for use in some procedures, for example an angioplasty procedure, wherein the so-called “land zone” distal to the treatment site may not be long enough to receive both the filter and the occluder.

Another catheter featuring a combination of filter and occluder elements is the subject of U.S. Pat. No. 6,994,718 B2, commonly assigned to the assignee of the invention herein. In the catheters of the '718 patent, a filter surrounds an inflatable occlusion balloon, which requires an elongate lumen to provide fluid communication between the balloon and an inflation/deflation system outside the patient. Catheters having occlusion balloons must also be carefully designed to avoid fluid leaks, especially from the balloon itself. The '718 patent also teaches an embodiment wherein a filter surrounds a non-inflatable occluder that is expandable by push-pull components in addition to those required to operate the filter.

A distal protection catheter that filters and occludes without a balloon-type occluder is the subject of U.S. Appl. Pub. No. US 2006/0155322 A1 to Sater et al., commonly assigned to the assignee of the invention herein. The distal protection device of the Sater publication features a braided mesh component that is selectively transformable between a filter configuration and an occluder configuration either by mechanical or thermal operation.

However, a need still exists in the art for an alternative distal protection device having the perfusion benefit of a filter while also selectively providing the benefit of an occluder for complete particle capture.

### SUMMARY OF THE INVENTION

Accordingly, disclosed herein is a distal protection device for containing embolic debris in a body lumen that includes a combined filtration and occlusion mechanism positioned at a distal end thereof. In one embodiment, the distal protection device includes a tubular member having a lumen extending there through, a core wire slidably disposed within the lumen of the tubular member and a filtration and occlusion element having a filter component and a coil occluder component. The filter component has openings for allowing blood flow there through and a proximal port for receiving embolic debris, wherein a proximal end of the filter component is coupled to the distal end of the tubular member and a distal end of the filter comoponent is coupled to the core wire. The coil occluder component is slidably disposed within the lumen of the tubular member and has windings on a distal portion thereof. The windings of the coil occluder component are selectively stackable within the filter component to occlude flow through the filter component. When the windings are tightly stacked within the filter component, fluid flow through the filter component is substantially stopped, such that the filtration and occlusion element is in an occlusive configuration. In another embodiment, when the filtration and occlusin element is in an occlusive configuration, the windings are tightly stacked with the filter component such that the proximal port and proximal openings of the filter component are blocked. In another embodiment, one or more windings may be advanced into or withdrawn from an interior of the filter component to regulate a rate of blood flow there through.

In another embodiment, the distal protection device includes an elongate tubular member and a filtration and occlusion element. The filtration and occlusion element has a filter component having openings for allowing blood flow there through and a proximal port for receiving embolic debris, wherein a proximal end of the filter component is coupled to a distal end of the tubular member. The filtration and occlusion element also has a coil occluder component slidably disposed within a lumen of the tubular member and having windings on a distal portion thereof. The windings of the coil occluder component are selectively stackable within the filter component to deploy the filter component from a collapsed delivery configuration into a filter configuration. When the windings are tightly stacked within the filter component, fluid flow through the filter component is substantially stopped, such that the filtration and occlusion element is in an occlusive configuration. In another embodiment, when the filtration and occlusion element is in an occlusive configuration the windings are tightly stacked within the filter component such that the proximal port and proximal openings of the filter component are blocked. In another embodiment, one or more windings may be advanced into or withdrawn from an interior of the filter component to regulate a rate of blood flow there through.

A method of using the distal protection device in accordance with another embodiment of the present invention includes providing a distal protection device including a filtration and occlusion element having a filter component with a plurality of pores for allowing blood flow there through and a proximal port for receiving embolic debris and a coil occluder component having windings on a distal end thereof that are selectively stackable within the filter component. The distal protection device is tracked distal of a treatment site within a vessel, where the filter component is deployed from a collapsed configuration to a filtering configuration, such that a portion of an outer surface of the filter component is placed in apposition to a vessel wall. One or more windings of the coil occluder component are then advanced into the filter component to at least partially close the proximal pores and proximal port of the filter component. The windings may be tightly stacked within a proximal portion of the filter component to close the proximal pores and proximal port, such that the filtration and occlusion device is in an occlusive configuration. In a further embodiment, embolic debris that collects proximal to the filtration and occlusion device in its occlusive configuration is aspirated.

### BRIEF DESCRIPTION OF THE DRAWINGS

The foregoing and other features and advantages of the invention will be apparent from the following description of the invention as illustrated in the accompanying drawings. The accompanying drawings, which are incorporated herein and form a part of the specification, further serve to explain the principles of the invention and to enable a person skilled in the pertinent art to make and use the invention. The drawings are not to scale.

FIG. 1 illustrates a side view of a distal protection device in accordance with an embodiment of the present invention.

FIG. 2 illustrates a side view of a distal portion of the distal protection device of FIG. 1 with a filtration and occlusion element in a collapsed configuration.

FIG. 3 illustrates a longitudinal sectional view of a rotateable connection between a core wire and a distal end of a filter component in accordance with another embodiment of the present invention.

FIG. 4 illustrates a longitudinal sectional view of the distal portion of the distal protection device of FIG. 2 with the filtration and occlusion element in a filter configuration.

FIG. 5 illustrates a longitudinal sectional view of the distal portion of the distal protection device of FIG. 2 with the filtration and occlusion element in an occlusive configuration.

FIG. 6 illustrates a longitudinal sectional view of a filtration and occlusion element in a filter configuration in accordance with another embodiment of the present invention.

FIG. 7 illustrates a longitudinal sectional view of a filtration and occlusion element in a filter configuration in accordance with another embodiment of the present invention.

FIGS. 8 and 9 illustrate a coil occluder component in accordance with an embodiment of the present invention.

FIG. 10 illustrates a filtration and occlusion element in accordance with another embodiment of the present invention.

FIG. 11 illustrates a distal portion of a filtration and occlusion element in an occlusive configuration in accordance with another embodiment of the present invention.

### DETAILED DESCRIPTION OF THE INVENTION

Specific embodiments of the present invention are now described with reference to the figures, wherein like reference numbers indicate identical or functionally similar elements. The terms “distal” and “proximal” are used in the following description with respect to a position or direction relative to the treating clinician. “Distal” or “distally” are a position distant from or in a direction away from the clinician. “Proximal” and “proximally” are a position near or in a direction toward the clinician.

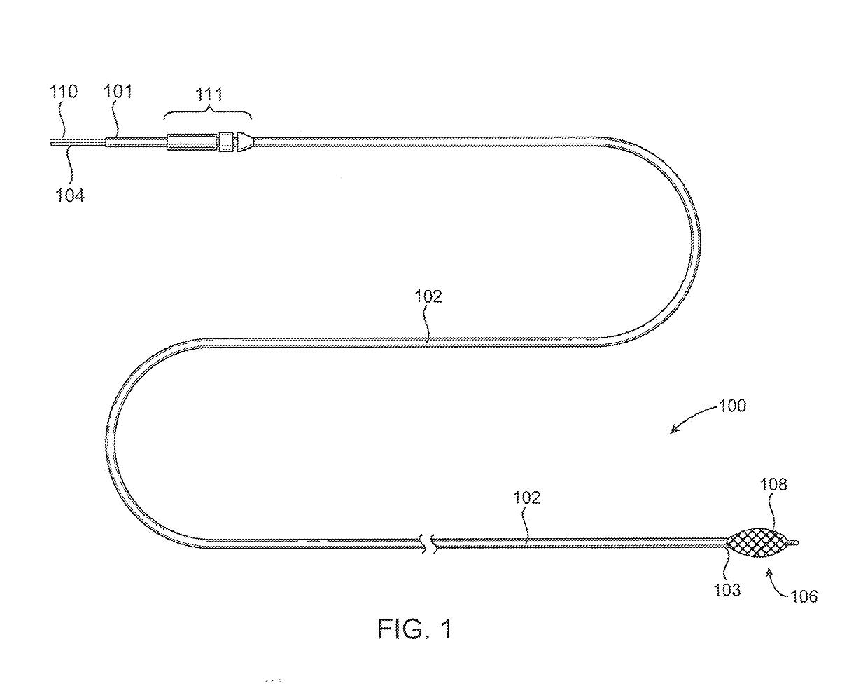

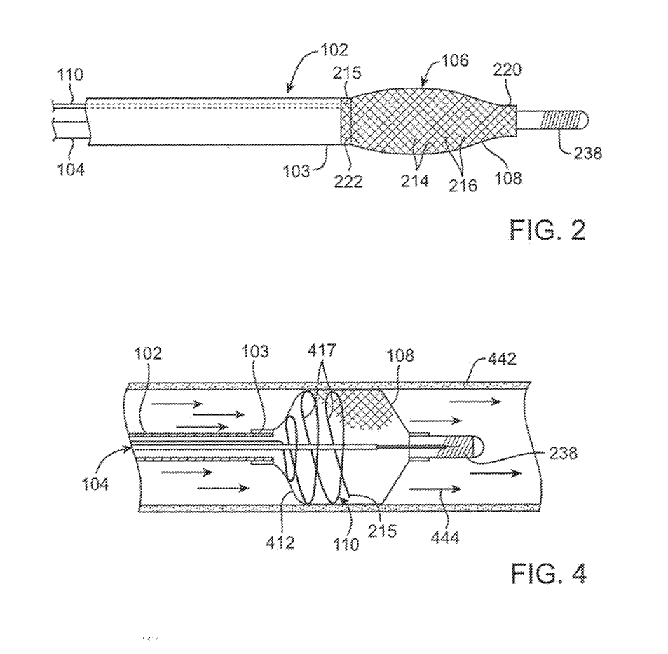

The present invention is a temporary distal protection device 100 for use in minimally invasive procedures, such as vascular interventions or other procedures, where the practitioner desires to capture and remove embolic material that may be dislodged during the procedure. As shown in FIGS. 1 and 2, distal protection device 100 includes an elongate tubular member, or catheter shaft 102, a core wire 104 slidably extending there through, and a hub 111. Core wire 104 extends within a lumen of tubular member 102 from a proximal end 101 to a distal end 103 thereof. A filtration and occlusion element 106 for selectively providing filtering or occlusion of embolic debris has a filter component 108 joined to distal end 103 of tubular member 102 and core wire 104, as described below, and a coil occluder component 110 that slidably extends within the lumen of tubular member 102 from proximal end 101 to distal end 103.

FIG. 2 illustrates a side view of filtration and occlusion element 106 in a collapsed configuration according to an embodiment of the present invention. Coil occluder component 110 of filtration and occlusion element 106 is shown extending within tubular member 102 in a straightened, pre-deployment configuration with a distal end 215 situated proximate filter component 108. Coil occluder component 110 is an elongate slender shaft slidable within the lumen of tubular member 102 and therefore is not attached along its length to tubular member 102. Filter component 108 of filtration and occlusion element 106 has a proximal end 222 attached to distal end 103 of tubular member 102 and a distal end 220 axially secured to core wire 104. Filter ends 220, 222 may be soldered, spot welded, laser welded or secured using bonding sleeve or adhesive to tubular member 102 or core wire 104, respectively, as would be apparent to one skilled in the relevant art. Although filter component 108 is shown attached to the outer surface of tubular member 102, filter component 108 may, alternatively, be butt-joined or attached to the inner surface at tubular member distal end 103. In FIG. 2, filter component 108 is collapsed about core wire 104 and has an outer diameter that is close in size to the outer diameter of tubular member 102. The low profile of the collapsed configuration of filter component 108 allows distal protection device 100 to be navigated through blood vessels and across narrow stenoses.

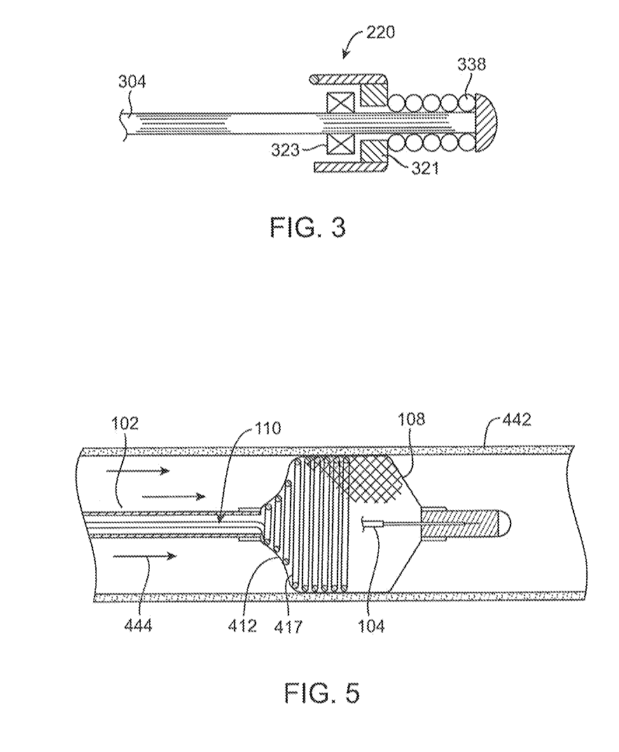

FIG. 3 illustrates an alternate arrangement for joining filter component distal end 220 to a core wire 304. In this embodiment, filter component distal end 220 is affixed to a cylindrical collar or bearing 321, such that core wire 304 may rotate relative to the filter component. Filter component distal end 220 is held in its axial position relative to core wire 304, proximally be a stop 323 and distally be a flexible tip 338.

Core wire 104 is a long, thin flexible wire similar to medical guidewires and core wires known in the art that is slidably disposed through tubular member 102 and filter component 108. Core wire 104 may be made from a metal, such as nitinol, stainless steel, or cobalt-chromium superalloy wire. In an embodiment of the present invention, core wire 104 may be tapered at its distal end and/or may include one or more core wire sections of different materials. Core wire 104 may be centerless-ground to have several diameters in its profile in order to provide regions of different stiffnesses with gradual transitions there between. Core wire 104 has a proximal end that extends outside of the patient from proximal end 101 of tubular member 102. Core wire 104 may also include a coiled tip portion, such as coiled tip portion 238 shown in FIG. 2, or may include a flexible tip, such a flexible tip 338 shown in FIG. 3, that is formed from a round or flat coiled wire of stainless steel and/or one of various radiopaque alloys, such as platinum tungsten, as is well known to those of skill in the art of medical guidewires.

Tubular member 102 is a long, hollow tube that is flexible enough to navigate the tortuous pathways of the cardiovascular system while being longitudinally incompressible enough to be pushed through the vasculature. Tubular member 102 may include a thin-walled, tubular structure of a metallic material, such as stainless steel, nitinol, or a cobalt-chromium superalloy. Such metallic tubing is commonly referred to as hypodermic tubing or a hypotube. Metallic tubing formed from other alloys, a disclosed in U.S. Pat. No. 6,168,571, which is incorporated by reference herein in its entirety, may also be used in the tubing of the present invention. In the alternative, tubular member 102 may include tubing made from a thermoplastic material, such as polyethylene block amide copolymer, polyvinyl chloride, polyethylene, polyethylene terephthalate, polyamide, or a thermoset polymer, such as polyimide.

If a polymeric material is utilized for tubular member 102, optionally, a layer of a stiffer reinforcing material may be added to or embedded within the main material of tubular member 102 for a portion or the entirety thereof enhance the longitudinal stiffness of distal protection device 100. For example, a braid of metal or polymeric filaments could be included. In addition, a coating may be applied to the outer surface of tubular member 102 so that distal protection device 100 may slide more easily through a vessel. In addition, the inner surface of tubular member 102 and/or the outer surfaces of core wire 104 and/or coil occluder component 110 may include a coating to reduce sliding friction of core wire 104 and coil occluder component 110 within tubular member 102. In another embodiment, tubular member 102 may be a polymeric extrusion having dual, side-by-side lumens, wherein one lumen is dedicated to core wire 104 and the other lumen is dedicated to coil occluder component 110.

In other embodiment of the present invention, tubular member 102 may be constructed of multiple shaft components of varying flexibility to provide a gradual transition in flexibility as the shaft extends distally. Such a shaft arrangement is disclosed in U.S. Pat. No. 6,706,055, which is incorporated by reference herein in its entirety. In addition, a liner or axial bearing (not shown) as disclosed in the '055 patent may be utilized between during expansion and collapse of filter component 108.

Filter component 108 is a tubular braided filter constructed of a plurality of wires or filaments 214 that are woven together to form the filter with a plurality of openings, or pores 216 and one or more proximal ports or inlets 412 for admitting embolic debris into the interior of filter component 108. Filter component 108 is designed so that pores/openings 216 are small enough to trap or filter particulate debris while allowing blood and smaller blood components to flow there through, as indicated in FIG. 4 by blood flow arrows 444. Filter components 108 is sized and shaped such that when it is fully deployed, as in FIG. 4, an outer surface of filter component 108 will contact the inner surface of the blood vessel wall into which it is placed to prevent potentially contaminated blood from flowing around the distal protection device.

Filaments 214 of filter component 108 may be made from any biocompatible material known in the art. For example, filter component 108 may be constructed of stainless steel, a cobalt-based super alloy, shape-memory alloys, such as nitinol, or thermoplastic or thermoset polymers. Optionally, radiopaque markers (not shown) may be placed on proximal and distal ends 222, 220 of filter component 108 is aid in fluoroscopic observation during manipulation thereof. Alternatively, fluoroscopic visualization of filter component 108 may be enhanced when at least one of the filaments 214 includes a wire having enhanced radiopacity compared to conventional non-radiopaque wires suitable for braiding filter component 108. Filaments 214 having enhanced radiopacity may be made of, or coated with a radiopaque metal, such as gold, platinum, tungsten, alloys thereof, or other biocompatible metals having a relatively high X-ray attenuation coefficient compared with stainless steel or nitinol. One or more filaments 214 having enhanced radiopacity may be inter-woven with non-radiopaque wires, or all wires that form filter components 106 may have the same enhanced radiopacity.

Alternatively, one or more of braiding wires/braid filaments 214 may include a composite wire having a radiopaque core and non-radiopaque layer or casing. Such coaxial, composite wires are referred to as DFT (drawn-filled-tube) wires in the metallic arts, and filters utilizing such wires are disclosed in U.S. Pat. No. 6,866,677 B2 that is incorporated by reference herein in its entirety.

In an alternate embodiment, filter component 108 may be formed from a suitable mesh, perforated membrane, or other porous material that collects embolic debris while permitting fluid to flow there through, such as blood flow sufficient for perfusion of body tissues. Such mesh filters and braided filters are disclosed in U.S. Pat. No. 6,346,116 that is incorporated by reference herein in its entirety.

FIG. 4 illustrates filter component 108 of filtration and occlusion element 106 in a radially expanded or filtering configuration, wherein a portion of filter component 108 is in apposition to vessel wall 442. In accordance with an embodiment of the present invention, filter components 108 may be transformable between its collapsed and expanded configurations by relative movement between its ends 220, 222, e.g., by holding tubular member 102 stationary and advancing/withdrawing core wire 104 relative thereto, filter component ends 220, 222 are bought closer together or moved father apart according to whether filter component 108 is being expanded or collapsed.

Coil occluder component 110 is shown in FIG. 4 advanced within the interior of filter component 108 with several windings 417 expanded against an interior surface of filter component 108. A distalmost winding 417 is positioned to expand within filter component 108 at a point along the contracting interface between the outer surface of filter component 108 and vessel wall 442.

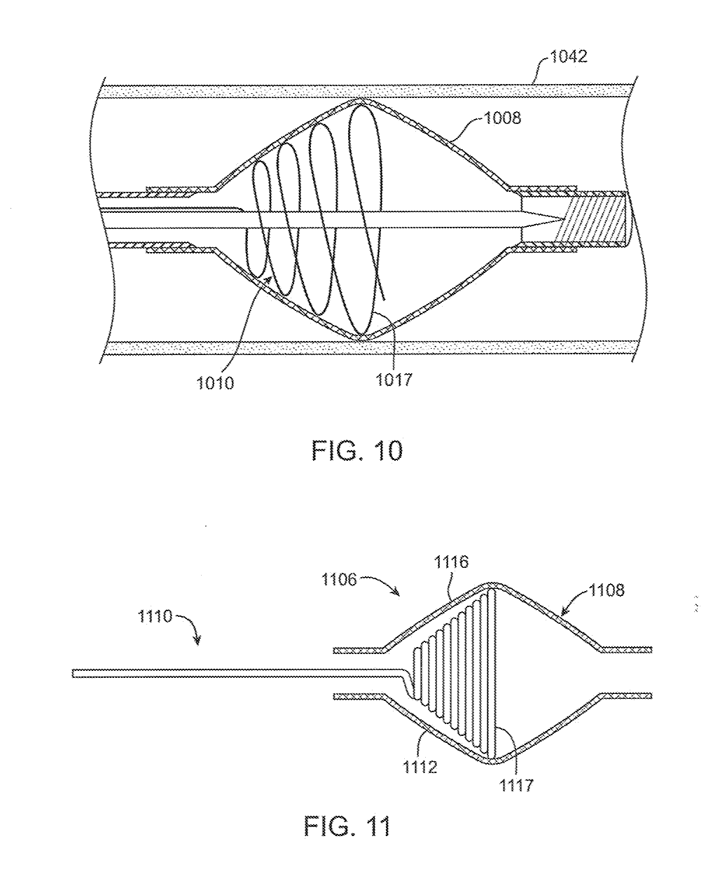

In an embodiment shown in FIG. 10, a distalmost winding 1017 of coil occluder component 1010 is expanded at a point within filter component 1008 where a diameter of filter component 1008 is at a maximum and in contact with vessel wall 1042. The maximum inner diameter of filter component 1008 acts as a winding stop to assure proper positioning and to prevent distal movement of windings 1017 when they are stacked within filter component 1008. The stacked arrangement of windings 1017 may appear similar to the stacked arrangement of windings 417 shown in FIG. 5, wherein windings 417 are stacked against adjacent turns of coil occluder component 110 and some or all of the turns are also disposed in apposition to the inner surface of filter component 108.

In another embodiment, distal end 215 of distalmost winding 417 may include a small outward bend or hook (not shown) that acts as a winding stop when engaged with one of pores 216 along a sidewall of filter component 108 to assure proper positioning and prevent distal movement of windings 417 of coil occluder component 110. In another embodiment, distal end 215 of windings 417 may be a disc, e.g., disc 646 discussed below, that is rotatable about core wire 104 and is properly positioned so as to be prevented from distal movement within filter component 108 by a stop ring, e.g., stop 323 discussed above, that is fixedly attached to core wire 104. In another embodiment, a radiopaque marker (not shown) may be placed on distal tip 215 of coil occluder component 110 to aid in the proper positioning of distalmost winding 417.

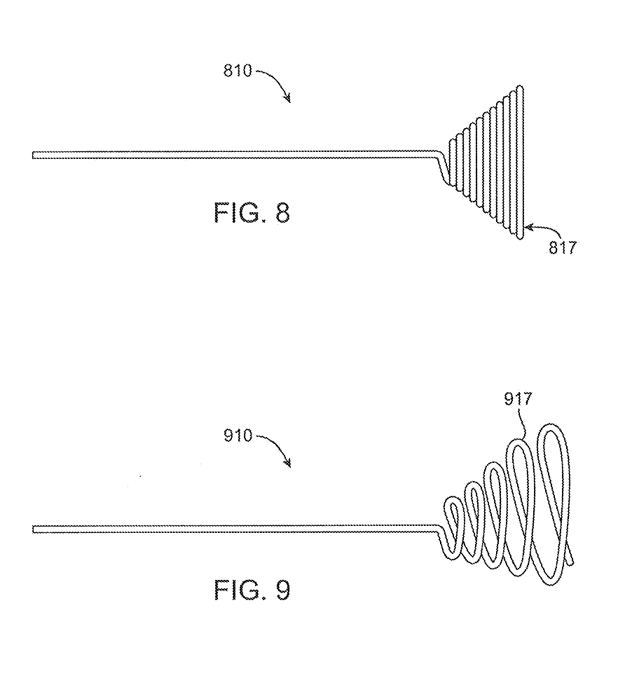

In the configuration of coil occluder component 110 shown in FIG. 4, there is an insufficient number of windings 417 advanced and expanded within filter component 108 to interfere with blood flow through pores 216 or proximal inlets 412, such that filtering still occurs. However, coil occluder component 110 is shown in FIG. 5 fully advanced within the interior of filter component 108 with windings 417 stacked-up against one another and also disposed in apposition to the inner surface of filter component 108 to block proximal inlets 412 and proximal pores 216 of filter component 108 and thereby prevent blood flow through filtration and occlusion element 106. As such, FIG. 5 illustrates filtration and occlusion element 106 of FIG. 2 within a body lumen in an occlusive configuration, wherein embolic debris is prevented from entering filter component 108 and instead is congregated proximal of filtration and occlusion element 106, where it may then be aspirated from the blood vessel as needed. In an alternate embodiment illustrated in FIG. 11, a filtration and occlusion element 1106 is shown in an occlusive configuration having a coil occluder component 1110, similar to coil occluder component 810 of FIG. 8, in a fully stacked arrangement within a filter component 1108. In its fully stacked arrangement, fewer than all windings 1117 of coil occluder component 1110 touch filter component 1108, such that windings 1117 do not actually block proximal inlets 1112 and proximal pores 1116 but nonetheless substantially stop blood flow through filtration and occlusion element 1106.

During an interventional procedure, any number of windings 417 may be selectively advanced within or withdrawn from the interior of filter component 108 to effectively “open or close” proximal inlets 412 and/or proximal pores 216, thereby achieving a desired flow rate, i.e., rate of perfusion or blood flow, through filtration and occlusion element 106. Upon completion of the interventional procedure, windings 417 of coil occluder component 410 may remain within filter component 408 to be collapsed and removed therewith. Alternatively, windings 417 may be withdrawn from filter component 208 such that coil occluder component 210 is returned to its straightened configuration within tubular member 102 for removal with, or in advance of distal protection device 100. In an embodiment, prior to removal of filter component 408, a sufficient number of windings 417 may be introduced within filter component 408 to “close” proximal inlets 412 such that when filter component 408 is collapsed, windings 417 act as a closure device preventing the escape of particulate from filter component 208 during removal of distal protection device 100 from the vasculature.

Windings 417 of coil occluder component 110 may be formed of a shape memory alloy, such as nitinol, that is pre-set into a coil configuration. In an embodiment shown in FIG. 8, coil occluder component 810 is formed from a shape memory alloy and the distal end thereof includes coil windings 817 being set in an ogival or generally conical shape having a distal-to-proximal taper, such as a flared profile. The flared configuration of coil windings 817 may substantially match an expanded profile of a proximal portion of a filter component. In use, windings 817 of coil occluder component 810 will automatically form the tightly stacked, impervious arrangement shown in FIG. 8 as they are advanced within a filter component and, as such, do not require a winding stop and/or engagement with the filter component to prevent distal movement of the distalmost winding 817. Alternatively, the coil windings formed in the distal end of the occluder component may be set in an ogival or generally conical shape having a proximal-to-distal taper (not shown), i.e., the opposite of the flared profile shown in FIG. 8. Such a proximal-to-distal tapered configuration of coil windings may substantially match an expanded profile of a distal portion of a filter component. As discussed below with respect to the embodiment shown in FIG. 11, a tightly stacked, impervious arrangement of coil windings disposed anywhere within a filter component may occlude blood flow through the filter component.

FIG. 9 illustrates another embodiment of coil occluder component 910 that includes windings 917 formed in a loosely stacked tapered profile. In use, windings 917 become tightly stacked within a filter component when distalmost winding 917 abuts a winding stop as discussed above and a sufficient number of windings 917 are introduced with the filter component to cause each to abut one another. As discussed with reference to the embodiments disclosed above, a winding stop or engagement with the filer component may be beneficial in assuring proper placement and preventing distal movement of windings 917 of coil occluder component 910 within the filter component as the windings are tightly stacked such that the filtration and occlusion element is transformed from a filter configuration into an occlusive configuration.

Distal protection device 100 traverses the vascular anatomy in its collapsed configuration, as shown in FIG. 1. In an interventional procedure, such as PTCA, filtration and occlusion element 106 of distal protection device 100 is normally tracked distal of a treatment site to contain embolic debris that might be created during the procedure. Filter component 108 is expanded/deployed distal of the treatment site by withdrawing core wire 104 proximally through tubular member 102 to move filter ends 220, 222 closer together, thereby causing transformation of filter component 108 into the expanded configuration shown in FIG. 4. The expansion of filter component 108 stops when the filer is in apposition with the vessel wall of the patient. Surface contact is preferably maintained around the entire vessel lumen to prevent any emboli from slipping past filter component 108 during the procedure.

At any time during the interventional procedure, a clinician may change a perfusion rate through filter component 108 by introducing or withdrawing one or more windings 417 of coil occluder component 110 into or out of filter component 108. An clinician may also transform the filtration and occlusion element 106 from a filter configuration shown in FIG. 4 into the occlusive configuration shown in FIG. 5 by advancing a sufficient number of windings 417 into filter component 108 so that they form an impervious stack of windings 417 that effectively spans the inner diameter of filter component 108. In a tightly stacked arrangement, windings 417 may effectively close proximal pores 216 and port 412 by contacting filter component 108. However, to transform a filtration and occlusion element into an occlusive configuration, it is not necessary for windings of the occluder component to contact a proximal portion of filter component and block proximal pores and/or ports. As illustrated in FIG. 11, a tightly stacked arrangement of windings 1117 disposed anywhere within filter component 1108 may occlude blood flow through filter component 1108 with as few as one winding 1117 contacting filter component 1108 at a maximum diameter of filter component 1108 where it is in apposition to the vessel wall.

When an intravascular treatment is complete, any embolic debris collected proximal of filtration and occlusion element 106 may be aspirated, as by use of a separate aspiration catheter (not shown). The, filter component 108, which may contain embolic debris, is collapsed and removed from the patient. To help prevent release of captured particulate, windings 417 of coil occluder component 110 can be left within filter component 108 during collapse and removal thereof. In an embodiment, filter component 108 is mechanically collapsed by the push-pull mechanism previously discussed above. Accordingly, as filter comment 108 is drawn down by distal movement of the core wire 104 relative to tubular member 102, the collapse of windings 417 within the proximal portion of filter component 108 assures proximal inlet 412 is at least partially covered, reducing the possibility that particulate will be released during removal from the patient's vasculature.

As is well known in the art, components made of alloys having thermal shape-memory properties are capable of transforming from one shape to another simply by increasing the temperature of the components. For example, when nitinol is used, a component may be shaped and heat-treated so that it has a memorized shape when the material is in an austenite phase. After cooling, the material transforms into a martensite phase wherein the material can be deformed so that it retains a different shape. When the temperature of the material is increased to the austenite finish temperature Af (i.e., the temperature at which the transformation from martensite to austenite finishes upon heating) for the particular grade of nitinol, then the material returns to the austenite phase and the component will tend to return to the memorized shape. In embodiments of the present invention, a coil occluder component may be formed to have thermal shape memory properties such that the memorized shape includes coils or windings in a distal portion thereof. In use, a distal protection device having a filtration and occlusion element according to this embodiment of the present invention may be introduced within a body lumen such that a filter component is expanded within a landing zone distal to a treatment site. A coil occluder component in a straight configuration may then be tracked through the distal protection device, such that a distal end is positioned within an interior of the expanded filter component. Accordingly as the coil occluder component reaches a transformation temperature, the distal portion will transform from the straight configuration into the memorized coiled configuration, as coils/windings form on a distal portion thereof. In such an embodiment, self-forming of the coils/windings within the filter component may act to distally pull remaining coils/windings of the coil occluder component from a lumen of the proximal tubular member into the filter components.

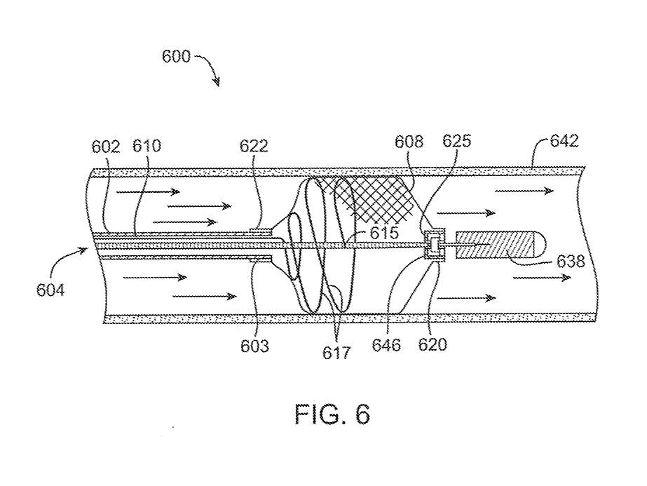

FIG. 6 illustrates distal protection device 600 in accordance with another embodiment of the present invention, wherein braided filter component 608 is shown in an expanded configuration with a portion of filter component 608 in apposition to vessel wall 642. Distal protection device 600 has a construction similar to distal protection device 100 described above, which includes an elongate tubular member 602 with a slidable core wire 604 extending there through and having filter component 608 attached at a distal end 603 thereof. Core wire 604 has a flexible distal tip 638 that extends distally of filter component 608.

A proximal end 622 of filter component 608 is fixedly attached about distal end 603 of tubular member 602. However, as distinguished from the construction of distal protection device 100, a distal end 620 of filter component 608 is rotatably attached about core wire 604 near core wire distal tip 638. Filter component 608 includes a distal mounting collar 625 rotatably mounted about disc 646, which is fixedly mounted about core wire 604. Disc 646 prevents distal mounting collar 625 from sliding proximally along core wire 604. Optionally, disc 646 may also prevent distal mounting collar 625 from sliding distally along core wire 604. In distal protection device 600, core wire 604 rotates freely within filter component 608. Alternatively, the rotateable connection between filter component distal end 620 and core wire 604 may be achieved in a manner that is similar to that shown in FIG. 3.

Further in the embodiment of FIG. 6, a distal end 615 of coil occluder component 610 is fixedly attached to core wire 604 at approximately a midpoint of filter component 608. As such, windings 617 of coil occluder component 610 may be aided in unwinding within the interior of filter component 608 by rotating core wire 604. The materials for the various components of distal protection device 600 may be the same as those previously described.

In the configuration of coil occluder component 610 shown in FIG. 6, there is an insufficient number of windings 617 advanced and expanded within filter component 608 to interfere with blood flow through filter component 608, such that filtering still occurs. However, similar to the configuration shown in FIG. 5, coil occluder component 610 may be fully advanced within the interior of filter component 608 whereby windings 617 stack-up against one another to block blood flow through filter component 608 and to transform the filtration and occlusion element from a filter configuration into an occlusive configuration.

As discussed above, a filter component in accordance with the present invention may be transformable between its collapsed and expanded configurations by relative movement between its ends. In various embodiments, such movement may be accomplished by a filter guidewires mechanism similar to that shown in any of the filter guidewires disclosed in U.S. Pat. No. 6,706,055, U.S. Pat. No. 6,818,006 and U.S. Pat. No. 6,866,677, which are incorporated by reference herein in their entireties. Alternatively, a filter component in accordance with the present invention may be deployed and/or retrieved via a sheath catheter, such as by the method and apparatus disclosed in U.S. Pat. No. 6,059,814, which is incorporated by reference herein in it entirety, or the '116 patent previously incorporated by reference. The transformation of the filter component may be impelled by external mechanical means alone or by self-shaping memory (either self-expanding or self-collapsing) within the filter. Preferably, filter component 108 is self-expanding, meaning that filter component 108 has a mechanical memory to return to the expanded, or deployed configuration. As previously discussed with respect to filters formed by braided wires, such mechanical memory can be imparted to the metal that forms filter component 108 by thermal treatment to achieve a spring temper in stainless steel, for example, or to set a shape memory in s susceptible metal alloy, such as nitinol. In such an embodiment of the present invention, it is preferable that at least the majority of braiding wires forming filter component 108 be capable of being heat treated into the desired filter shape, and such wires should also have sufficient elastic properties to provide the desired self-expanding or self-collapsing features.

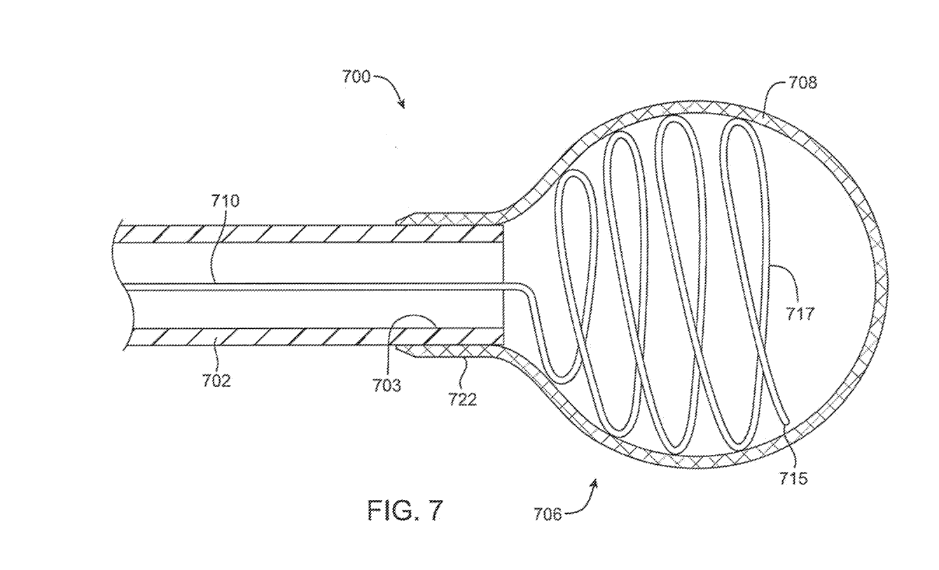

FIG. 7 illustrates a sectional view of a filtration and occlusion element 706 in a filter configuration of a distal protection device 700 in accordance with another embodiment of the present invention. Distal protection device 700 includes an elongate tubular member 702, as described above with reference to the previous embodiments, having a distal end 703 attached to a proximal end 722 of a filter component 708. A coil occluder component 710 slidably extends with a lumen of tubular member 702 with windings 717 advanced and expanded within an interior of filter component 708. In this embodiment, the expansion of windings 717 within the interior of filter component 708 mechanically expands/deploys filter component 708 into apposition with a vessel wall, eliminating the need for a push-pull mechanism and attendant structure. As such, a material for windings 717 must have sufficient strength to expand and support filter component 708, and in an embodiment may be any material specified for the coil occluder components of the previous embodiments by windings 717 may be of a thicker dimension to provide the required strength. As in the embodiments described above, additional windings 717 of coil occluder component 710 may be advanced and expanded within filter component 708 to control a rate of perfusion and/or to block blood flow through filtration and occlusion device 706. Filtration and occlusion device 706 is collapsed for removal from a patient's vasculature by withdrawal of windings 717 from filter component 708 and/or through the use of a sheath catheter, as would be apparent to one of ordinary skill in the art. In various embodiments, filter component 706 may be a perforated membrane, a tubular braided filter or a mesh filter, and may be made of a shape memory alloy to be at least partially self-expanding or self-collapsing.

While various embodiments according to the present invention have been described above, it should be understood that they have been presented by way of illustration and example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the appended claims and their equivalents. It will also be understood that each feature of each embodiment discussed herein, and of each reference cited herein, can be used in combination with the features of any other embodiment. All patents and publications discussed herein are incorporated by reference herein in their entirety.

What is claimed is:

1. A distal protection device for containing embolic debris in a body lumen, the distal protection device comprising:
an elongate tubularly member having a lumen extending from a proximal end to a distal thereof;
an elongate core wire slidably disposed within the lumen of the tubular member; and
a filtration and occlusion element including:
a filter component having openings for allowing blood flow there through and a proximal port for receiving embolic debris, wherein a proximal end of the filter component is coupled to the distal end of the tubular member and a distal end of the filter component is coupled to the core wire, and
an elongate coil occluder component slidably disposed within the lumen of the tubular member and having windings on a distal portion thereof, wherein the windings of the coil occluder component are selectively stackable within the filter component to occlude blood flow through the filter component.

2. The distal protection device of claim 1, wherein when the windings are tightly stacked within the filter component the filtration and occlusion elements is in an occlusive configuration.

3. The distal protection device of claim 2, wherein the occlusive configuration the proximal port and proximal openings of the filter component are blocked by the windings of the coil occluder component.

4. The distal protection device of claim 1, wherein one ore more windings may be advanced into or withdrawn from an interior of the filter component to regulate a rate of blood flow there through.

5. The distal protection device of claim 1, wherein relative longitudinal movement between the proximal and distal ends of the filter component transforms the filter component between a collapsed configuration and a filter configuration.

6. The distal protection device of claim 1, wherein the filter component is a tubular braided filter.

7. The distal protection device of claim 6, wherein the tubular braided filter comprises a shape-memory alloy.

8. The distal protection device of claim 1, wherein the filter component distal end is rotatably coupled about the core wire.

9. The distal protection device of claim 8, wherein a distal end of the coil occluder component is fixedly attached to the core wire such that rotation of the core wire feed windings into or withdraws windings from the filter component.

10. The distal protection device of claim 1, wherein the coil occluder component comprises a shape-memory alloy.

11. The distal protection device of claim 10, wherein the windings of the coil occluder component are set in a tapered configuration.

12. A distal protection device for containing embolic debris in a body lumen, the distal protection device comprising:
an elongate tubular member having a lumen extending from a proximal end to a distal thereof; and
a filtration and occlusion element including:
a filter component having openings for allowing blood flow there through and a proximal port for receiving embolic debris, wherein a proximal end of the filter component is coupled to the distal end of the tubular member, and
an elongate coil occluder component slidably disposed within the lumen of the tubular member and having windings on a distal portion thereof, wherein the windings of the coil occluder component are selectively stackable within the filter component to deploy the filter component when the filtration and occlusion element is transformed into a filter configuration and to occlude blood flow through the filer component when the filtration and occlusion element is in an occlusive configuration.

13. The distal protection device of claim 12, wherein the windings are tightly stacked within the filter component when the filtration and occlusion element is in the occlusive configuration.

14. The distal protection device of claim 13, wherein the occlusive configuration the proximal port and proximal openings of the filter component are blocked by the windings of the coil occluder component.

15. The distal protection device of claim 12, wherein one or more windings may be advanced into or withdrawn from an interior of the filter component to regulate a rate of blood flow there through.

16. The distal protection device of claim 12, wherein the coil occluder component comprises a shape-memory alloy.

17. The distal protection device of claim 16, wherein the windings of the coil occluder component are set in a tapered configuration.

18. A method of using a distal protection device comprising:
providing a distal protection device including a filtration and occlusion element having a filter component with a plurality of pores for allowing blood flow there through and a proximal port for receiving embolic debris and a coil occluder component having windings on a distal end thereof that are selectively stackable within the filter component;
locating the filtration and occlusion element within a blood vessel;
deploying the filter component from a collapsed configuration to a filtering configuration wherein a portion of an outer surface of the filter component is placed in apposition to a vessel wall; and
advancing one or more windings of the coil occluder component into the filter component to at least partially occlude the filter component.

19. The method of claim 18, further comprising:
tightly stacking the windings within the filter component such that the filtration and occlusion element is in an occlusive configuration.

20. The method of claim 19, wherein the occlusive configuration the windings of the coil occluder component block the proximal pores and proximal port of the filter element.

21. The method of claim 18, further comprising:
aspirating embolic debris contained proximal to the filtration and occlusion device.

22. The method of claim 18, further comprising:
advancing one or more windings into or withdrawings one or more windings from an interior of the filter component to regulate a rate of blood flow there through.
